(19) United States
(12) Patent Application Publication
(10) Pub. No.: US 2009/0112261 A1
(43) Pub. Date: Apr. 30, 2009

(54) MINIMALLY INVASIVE SPINE INTERNAL FIXATION SYSTEM

(76) Inventor: Richard J. Barry, Vacaville, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/926,210

(22) Filed: Oct. 29, 2007

(51) Int. Cl. A61B 17/70; A61B 17/56; A61B 17/04; A61B 19/00; A61B 17/58

(52) U.S. Cl. 606/246; 606/99; 606/301; 606/264; 128/898

(57) ABSTRACT

A minimally invasive modular system is disclosed for stabilizing the anterior column of the spine through an anterior or lateral approach. Bone anchors are implanted into the vertebral bodies of adjacent vertebrae and serve as a foundation for a fixation system. The anchors comprise an interface surface that allows an interconnecting link to be secured to the anchors in a low-profile manner with minimum protrusion beyond the vertebral members. The link may be secured to the bone anchors to affect a compressive interlocking force between the bone anchors and vertebrae.

### CROSS-REFERENCE TO RELATED APPLICATIONS

Not Applicable

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to bone implant systems and methods, and more particularly to bone anchor systems and methods for stabilizing adjacent vertebrae.

2. Description of Related Art

There are a number of conditions in the spine which require a surgeon to stabilize or reinforce the spine with fixation devices applied anterior or lateral aspects of the vertebrae.

Indications for fusion of the spine include trauma, tumor, infection, and; most commonly, the consequences of degenerative change: all conditions which may produce progressive instability or compression of the neural elements and intractable pain. The most common indication for anterior fixation of the spine is in conjunction with a cervical fusion in which two or more vertebra in the neck are joined after excising the intervening intervertebral disc(s) and placing bone in the intervertebral joint(s). Similar surgical requirements exist for anterior or lateral stabilization of the thoracic and lumbar spine via a minimally invasive approach, for the purposes of clarity only the cervical spine surgical embodiments will be discussed here.

Current anterior cervical spine internal fixation systems attach two or more vertebrae with a plate connected to the frontal surface of the vertebra with multiple bone screws. The literature indicates that patient outcomes and the rate of successful arthrodesis of two or more levels of the cervical spine are enhanced with the use of anterior plate fixation applied to the vertebrae.

The current development trend in spine surgery is toward the performance of increasingly smaller incisions and less invasive surgical exposures to reduce collateral damage to normal soft tissues along the path of the surgical approach. In current practice, the surgical procedure to safely expose and apply a vertebral fixation plate to the anterior spine necessitates a larger incision than is required to address the pathology, primarily as a result of the size of the plate and the amount of tissue which must be disturbed to apply the fixation to the vertebrae.

Currently there exists no compact fixation system compact enough for use through the increasingly small minimally invasive anterior surgical approaches to the neck. There are multiple clinical indications for the performance of an anterior cervical fusion and well over 100,000 are performed each year in the United States alone. An important biomechanical goal of spinal instrumentation is the control of the forces across the vertebral motion segment for a period of time while healing tissues regain normal biomechanical strength. A solid osseous bridging fusion of the intervertebral joints at each level is critical to the success of anterior cervical spinal fusion surgery. Fusion rates are enhanced by controlling the forces and resultant motion across the intervertebral joints to be arthrodesed. For many years the success rate for anterior cervical fusion surgery has remained unchanged at approximately 85%.

The classic procedures for anterior fusion of the cervical spine require an approach to the front of the cervical spine through fairly long neck incisions which incise tissues from anteriolateral to the midline of the neck. The extensive wounds and long operative times of these procedures are frequently associated with complications arising directly from the extensive dissection and retraction required. The pressure produced by a typical soft-tissue retraction used to provide adequate surgeon visualization is a major cause of complications in anterior cervical spine surgery and yield unacceptably low patient satisfaction rates. The cost of failed spine surgery is enormous. Persistent pain, lost productivity and the disappointingly low success rate of revision surgery underscore the need for more effective primary cervical spine fusion surgery.

As a surgeon visualizes the anterior aspect of the cervical spine, there are a limited number of visual anatomic landmarks available. The initial portions of the cervical spine procedure quickly alter the appearance or position of these landmarks. Placement of anterior fixation to the cervical spine generally occurs toward the conclusion of a procedure when there are few visible undisturbed anatomic landmarks remaining to guide accurate placement of a plate fixation system of current design. Television image intensifier fluoroscopic visualization is frequently used during surgery to assist the surgeon in accurately affixing the anterior fixation system to the two or more vertebrae to be stabilized. A number of technical aspects of patient size and positioning may significantly limit the surgeon's ability to accurately place the fixation precisely in the desired position on the spine.

Previously described anterior cervical fixation implants have depended upon multiple screws placed into each vertebral body to achieve secure fixation. The dissection of tissues necessary to place screws laterally off the midline obscures the anatomic landmarks leading all too frequently misalignment off the midline. As bilateral vertebral fixation screw placement moves more laterally away from the midline, the risk of damaging the underlying vertebral arteries running on either side of the vertebra increases substantially. Complications arising from spinal instrumentation and screw fixation, such as injury to underlying or adjacent soft tissues, fixation loosening, failure or migration, increase directly with the number of screws that must be placed to achieve a stable construct. Stresses applied to spine fusion implants which exceed the local instant yield strength of the bone surrounding the fixation screws produce localized progressive loosening and failure of the fixation of the implant, disrupting the healing revascularization essential for bone formation and leading to a non-union of the intended fusion of the motion segment.

During the surgeon's journey into the complex anatomy surrounding a patient's spine, he frequently uses instruments referred to in general as retractors to hold back the patient's normal tissues from the surgeon's visual field. There are several types of surgical retraction systems in current use for anterior cervical spine surgery. Hand-held retractors are generally flat blade-like instruments used to pull the overlying soft tissues away from the line of dissection between tissues. This type of retraction requires the use of an assistant or occupies one of the surgeon's hands. Self-retaining retractors pull against themselves with two or more blades. These instruments quite often provide a large mechanical advantage during deployment while applying potentially injurious levels of force to the retracted soft tissues of the anterior neck. A pin or screw is frequently placed in the cervical vertebrae to provide a mechanism for skeletal distraction of the intervertebral joint to facilitate intervertebral graft or bone anchor placement. The vertebral pins may also be used as an anchor for hand-held or self-retaining soft tissue retractors in order to gain stability.

Current advances in spine surgical techniques involve the development of minimally invasive, tissue sparing approaches performed through tiny incisions under television image intensifier fluoroscopic guidance. New instrumentation systems should be able to be safely implanted with a minimum of soft tissue exposure or surgeon direct visualization.

For these reasons, it would be desirable to provide a new system to provide definitive fixation of the vertebral bodies of the cervical spine applied through a minimally invasive approach under fluoroscopic guidance.

At least some of these objectives will be met with the invention described herein.

### BRIEF SUMMARY OF THE INVENTION

The system and methods of the present invention provide a minimally invasive surgical solution to a number of biomechanical problems currently encountered by spine surgeons, particularly when operating on the anterior cervical spine.

The system and methods disclosed herein provide a high percentage, predictable spinal fusion using a safe, minimally invasive method to mechanically lock the motion segment in order to allow bone formation and fusion across the intervertebral joints with minimal tissue disruption. Vertebral fusion is achieved via instrumentation implanted through a 1 centimeter diameter portal.

The system of the present invention includes a vertebral fixation system to allow the surgeon to place secure bone anchors in the vertebral bodies early in the procedure, so as to take advantage of the undisturbed anatomic references visible on the front of the cervical spine. A fixation assembly is disclosed for stabilizing first and second adjacent vertebrae of the spine via fixation of the adjacent vertebrae. The fixation assembly attaches to the adjacent vertebrae through implanted bone anchors that are mechanically coupled to a fixation linkage with fasteners such as locking caps.

In one embodiment, vertebral the bone anchors are used as a foundation of a skeletal and soft tissue retraction system during the decompression, and which may then be linked together to provide anterior stabilization of the vertebral column. Radiation exposure for the patient and surgeon are limited by the use of fluoroscopy to confirm, rather than guide, accurate placement of the bone anchors.

The system includes a cervical spine surgical retraction system utilizing the bone anchor as a foundation. Retractors expand from their point of attachment deep in the surgical wound, on the anterior aspect of the cervical spine, providing the most efficient control and protection of deeper tissues while minimizing excessive retraction pressures on the more superficial tissues of the front of the neck.

A series of clear acetate radiographic overlay templates of different scales printed with outlines of the bone anchors may also be used for use during the surgeon's pre-operative planning process.

The system may also include a surgical kit having a range of size-specific drills, inserters, impactors and custom-length guide wires matched to the internal and external diameters of the instrumentation system.

An aspect of the invention is a method for anteriorly or laterally stabilizing two or more adjacent vertebrae of the spine. The method generally comprises placing a bone anchor into the subcortical bone of the anterior surfaces of the first vertebrae, and installing a bone anchor into the subcortical bone of the second vertebrae adjacent to the intervertebral joint between the two vertebrae. The bone anchors are coupled to a linkage that is configured to control the transmission of forces applied to each vertebrae and the motion of the articulation between adjacent vertebrae. The force of retraction of the immobilizing connecting linkage is applied to the vertebra through the bone anchor.

The method may further comprise forming a path and implanting the bone anchor into the subcortical bone of the anterior surfaces of the subsequent vertebrae, and installing an interlocking connector and locking fasteners to immobilize the subsequent intervertebral joint or joints.

In a preferred embodiment, forming the bone path is achieved by drilling a cylindrical bore into the subcortical vertebral bone from a midline anterior position, wherein a portion of each subcortical vertebral bone is removed to form grooves defining opposite ends of the cylindrical bore. More preferably, the cylindrical bore is drilled in a path substantially parallel to the planes of the vertebral end-plates.

Generally, the method is performed through a small skin incision, with image intensified fluoroscopic X-ray available to confirm anchor positioning in relation to the critical anatomy of the adjacent vertebrae. The visual anatomic landmarks, which are readily visible on the front of the cervical spine, may be used in conjunction with radiographic landmarks visualized on the screen of the fluoroscopic monitor. For example, size of the bone anchor may be determined by positioning a radiolucent bone anchor guide over the bone of the vertebrae and comparing the size on the image seen on the monitor.

In a preferred embodiment for cervical fusion, the bone path is formed by marking the visual anatomic midline of the target cervical vertebrae at a point between the bilateral Longus Colli muscles attached to the vertebrae on either side of the midline of the anterior cervical spine. Fluoroscopy is used to confirm midline localization and to establish a starting point location for the bone anchor along that line which is equidistant on a rostral-caudal line between the adjacent vertebral end-plates.

In a preferred method of the present invention, a guide wire is then installed at the location in the vertebral body to aid in positioning and guiding the instruments used in the procedure to the bone anchor site. Several cannulated instruments may be used to prepare the subcortical surfaces of vertebra.

For example, a blunt dilator may be positioned at the vertebral body by advancing it over the guide wire. The dilator is optimally configured to limit the depth of the bored bone path determining the size of the bone path. The blunt dilator is installed through the soft tissues of the anterior cervical musculature and down to the outer surface of the vertebral body.

A cylindrical bore is then drilled parallel to the opposing vertebral endplates, wherein the blunt dilator guides the path of the cylindrical bore. The drill used to create the bore may comprise a hollow core drill with an inside diameter matching that of the dilator such that the drill may be advanced over the dilator to score a kerf into the vertebral body. Typically, the bone path is drilled to a depth of approximately 75% of the vertebral body bone mass. A radiolucent bone anchor guide may be positioned at the vertebral body to measure the size of the bone path.

A bone anchor implant is then positioned at the bore and driven into the bore formed within the vertebral body. The bone anchor implants of the present invention are configured to withstand cross-axis translational stress displacement and long axis failure in the bone. When coupled to the mechanical linkage, the bone anchor inserts constrain motion of the vertebral body through broad diffusion of forces to the cortical and underlying subcortical bone. In one embodiment of the invention, a ring bone anchor is driven into a cylindrical kerf formed within the vertebral body. Alternatively, an oversized dowel is driven into the scored bone path to create an interference fit within the vertebrae to mechanically lock the articulation of the first vertebral body.

The bone anchors facilitate stabilization of two adjacent vertebrae of the spine, the adjacent vertebrae having first and second vertebral bodies each having opposing subcortical endplates and an intervening intervertebral disc which comprise the major articulation of the cervical spine. In such a configuration, when locked with a connecting link, the bone anchor provides a mechanical lock of the vertebral motion segment to resist the forces between the first and second vertebrae produced by the physiologic bending of the articulation across the intervertebral joint. The bone anchor may comprise a dowel or ring configured to be installed into a bone path into each adjacent vertebra, wherein the bone path bored into the vertebral subcortical bone.

The dowel-shaped bone anchor implant generally has an outer surface configured to create an interference fit within and between the cortical and subcortical bone of the vertebrae. Preferably, the outer surface of the dowel has an outer diameter sized to create the interference fit with the vertebral cortical and subcortical bone. The outer surface may preferentially be tapered to create the interference fit between the bone anchor and the osseous structure of the vertebral body vertebral bone, the taper emanating from a leading edge of the dowel.

In addition, the outer surface may be roughened to create the interference fit within the vertebrae. For example, the roughened external surface may comprise tantalum beads or other materials which are biocompatible with ingrowth of bone into the outer surfaces of the bone anchor. The roughened external surface may also be plasma sprayed with calcium hydroxyapatite crystals to enhance the immediate biologic fixation of the bone anchor with the surrounding cancellous bone. In addition, the roughened external surface may have undulations, teeth or ridges. Ideally, the external surface of the bone anchor is configured to interdigitate with the bone surrounding the joint surfaces providing an immediate mechanical interlock with the vertebral structure. Immediate mechanical immobilization of the highly cellular vertebral cancellous bone exposed by the drilling and implantation of the device is an important aspect of the bone anchor system, which is beneficial in protecting the cellular healing response which produces the bone formation and osseous fusion through the bone anchor and between adjacent vertebrae to promote osseous fusion.

The dowel is preferably cannulated along its insertional axis for installation into the vertebral body via a guide wire. The outside diameter of the dowel ranges from 6 mm to 12 mm, and the dowel ranges from 4 mm to 12 mm in length.

The bone anchor implant may also comprise a ring configured to be installed into a cylindrical kerf bone path bored into the vertebral body. The ring has an outer surface configured to create an interference fit with the surrounding bone of the kerf such that the ring constrains vertebral body motion through circumferential force diffusion when installed. The ring implant may be fabricated from titanium or similar metal, or a bioabsorbable material such as carbon fiber or a bioabsorbable polymer.

In some embodiments, the outer diameter of the ring outer surface may be sized accordingly to create the interference fit with the bone surrounding the kerf. The outer surface may also be roughened to create the interference fit. The roughened external surface may have one or more fenestrations, wherein the fenestrations promoting growth of cancellous bone surrounding the ring. The fenestrations may comprise circular holes or parallel columns running axially along the outer cylindrical surface. The fenestrations may also have raised edges at their perimeters, the raised edges forming an interference fit with the surrounding bone of the kerf. In addition, the outer surface may also have a tapered leading edge to facilitate installation of the ring.

In yet another aspect of the invention, a fixation assembly is disclosed for stabilizing first and second adjacent vertebrae of the spine via fixation of the adjacent vertebrae. The fixation assembly attaches to the adjacent vertebrae through implanted bone anchors that are mechanically coupled to a fixation linkage with locking caps.

The fixation assembly includes an internal fixation linkage which connects the bone anchors installed in the adjacent vertebrae to be treated. The bone anchors may be selected from of a series of scaled implants varying in 2 millimeter length increments. A plurality of series of bone anchors may be available to the surgeon to help deliver the desired biomechanical effect to the motion segment, be it compression, distraction or simply neutralization of the forces across the spine bone anchors.

In one aspect of the invention, the internal fixation linkage comprises a plate-like structure that acts to apply multi-directional (e.g. compressive saggital plane) forces to the adjacent vertebrae. The fixation linkage is three-dimensionally contoured to provide solid spatial control of two adjacent vertebrae through a secure single bone anchor within each vertebral body. The bone anchor is of a cross-sectional design optimized to resist relatively modest torque or side-bending forces, while strong enough in design to adequately control of saggital plane flexion-extension bending moments. The geometry of the angle of inclination of and nature of the interface between the internal fixation device and the vertebral body bone anchors allows the surgeon to determine the direction and magnitude of the forces applied to the spine. The elongate, multi-ply linkage of the present invention provides a less invasive and more effective minimally invasive mechanism for the anterior-lateral stabilization of adolescent idiopathic scoliosis.

The safety of an implanted spine fixation system is related to the robustness of the mechanical interconnect, i.e. the ultimate yield strength and fatigue resistance safety margins of the fixation system between bone anchors. Three-dimensional contouring of the plate-like member of the linkage allows the design to exhibit a range of desired elastic zone behaviors.

In one embodiment, secure fixation and positive force-transmission across the junctions between the bone anchors and the interconnecting linkage is provided by a locking rivet. The vertebral body bone anchor may have a fastening portion that is configured to be exposed outside the surface of the vertebral body. In a preferred embodiment, the fastening portion comprises a round, hollow post, which may also be threaded.

In one embodiment, the angle between the long axes of the vertebral bone anchor posts are measured. The surgeon may then use the measurements to select between a series of interconnecting linkages that correspond with the length and interface geometry needed to achieve the desired biomechanical effect for each individual patient.

The face of the bone anchor at the base of the post may comprise of a microscopically contoured surface of finger-like protuberances, e.g. hollow thin-walled squares. The bearing surfaces of the connecting portion of the internal fixation linkage may also have a similar micro-surface which interdigitates with the bone anchor and the under surface of a fastening member, e.g. locking rivet, lock nut, lock screw or the like fastener. As the rivet is compressed and locked to clamp the components together the thin-walled cells deform and intimately interdigitate with the protuberances, providing a strong cold-welded interface.

The rivet may comprise a ‘blind’ or ‘self-bucking’ rivet such as used in airframe construction and repair. The rivet length may be measured with a depth gauge. Sized in variable length, e.g. one millimeter, increments, the rivets are configured to provide compression to lock the elements of the assembly together. The shaft of the rivet may have a geometry which also controls any rotational moments between portions of the construct. Because pull-out failure is primarily a factor of by the ultimate yield point of bone and secondarily a factor of the tensile strength of the rivet, the deep locking portion of the rivet is optimized to augment and maximize effective pull-out strength by forming a wider flange within surrounding bone as it is being set. The instrumentation set may also include a tool to endoscopically unlock and remove a staple. The removal tool locks on to the face of the rivet and mechanically unlocks or drills out the central locking column releasing the interconnection.

In another aspect of the invention, a kit for preparing an bone anchor site for installing an implant between adjacent first and second vertebrae of a patient comprises a guide wire having a length configured to span to the bone anchor site from a location exterior to the patient, a cannulated dilator configured to be received on the guide wire such that the dilator can be positioned at the bone anchor site by advancing it along the guide wire, and a drill configured to bore a bone path at the bone anchor site. The drill has a hollow core with an internal diameter closely matching the outer diameter of the dilator such that the drill can be positioned at the bone anchor site by advancing it over the dilator.

In a preferred embodiment, the dilator is configured to be positioned at a centralized position on the surface of the vertebral body, and wherein the drill has an outer diameter configured to bore a kerf into the vertebral body. The drill will typically have an outer diameter ranging from 6 mm to 12 mm. Ideally, the dilator has a stop to limit the depth of the drill within the vertebral body.

The kit may also include a cannulated impactor configured to be received on the guide wire such that the impactor may be positioned at the bone anchor site by advancing it along the guide wire to drive the bone anchor into the bone path.

In a further aspect of the invention, a system is disclosed for installing a bone anchor at an implant site to stabilize two adjacent first and second vertebrae of a patient. The system generally has a guide wire having a length configured to span to the bone anchor site from a location exterior to the patient, wherein the guide wire assists in positioning the tools and bone anchor necessary to perform the installation. The system also has a cannulated dilator configured to be received on the guide wire such that the dilator can be positioned at the bone anchor site by advancing it along the guide wire, wherein the dilator has an outer diameter correlating to a physiological relationship between the desired bone anchor size and the size of the vertebrae to be used for fixation. The system further comprises a drill configured to bore a bone path at the bone anchor site, the drill having a hollow core with an internal diameter closely matching the outer diameter of the dilator such that the drill can be positioned at the bone anchor site by advancing it over the dilator.

Finally, the system includes a plurality of bone anchors sized to be installed in the scored bone path, and a fixation linkage that is configured to be received and connected to the bone anchors to constrain motion of the first and second adjacent vertebrae. The bone anchors, mechanical linkage, and locking cap implants are all configured to be advanced to the implant site via the guide wire.

In a preferred embodiment, the dilator is configured to be received at a point on the exterior surface of the vertebral body, and wherein the drill has an outer diameter configured to bore a kerf into the vertebral body. A cannulated impactor may also be used to drive the bone anchor into the bone path. In addition, the system may include a radiolucent bone anchor guide which can be positioned at the bone anchor site to determine the size of the bone anchor. The system may further include a radiographic overlay template configured to be placed over radiographic images of the patient to determine the size of the dilator during the surgeon's preoperative planning.

In another aspect of the invention, a retraction apparatus may be used to gently retract soft tissues adjacent to the spine so that the surgeon may see the vertebrae and intervertebral joint.

Another aspect comprises a system for stabilizing first and second body members in a patient. The system includes a first bone anchor configured to be installed in a bone path bored into the first body member, and a second bone anchor configured to be installed in a bone path bored into the second body member, wherein the first and second bone anchors each comprise a mounting surface. The system also has an interconnecting link configured to be installed on to the first and second bone anchors subsequent to installation of the first and second bone anchors, wherein the interconnecting link spans the first bone anchor and the second bone anchor when installed. The link comprises a first platform configured to mate with the mounting surface of the first bone anchor, and a second platform configured to mate with the mounting surface of the second bone anchor. First and second fasteners may be used to clamp the link on to the first and second bone anchors to restrain motion of the first body member with respect to the second body member.

In a preferred embodiment, first body member comprises a first vertebral body, and the second body member comprises second first vertebral body, wherein the link is configured to span the first and second vertebral bodies to fuse the first and second vertebral bodies.

In another embodiment, the first and second bone anchors comprise a distal section configured to interface with the bored bone path, and a proximal section comprising one or more mounting surfaces. Also a portion of the proximal section is configured to protrude from the vertebral body when installed.

The distal section of the bone anchor may comprise fenestrations to promote bony ingrowth from bone surrounding the bone path. The distal section of the bone anchors may also comprise longitudinal protrusions configured to create an interference fit with the surrounding bone path. The distal section may also be tapered to form an interference fit with the bone path.

In one embodiment, the bone anchor comprises a central channel running axially along the bone anchor. The central channel is configured to allow the bone anchor to be installed into the bone path over a guide wire. The distal section may be hollowed out to form a tube having a cavity.

In one mode, the first and second fasteners comprise a cap configured to be inserted through the central channel and lock to a surface of the cavity.

The platform and the mounting surface preferably comprise mating interlocking surfaces, wherein a first surface of the interlocking surfaces comprises an array of depressions, and wherein a second surface of the interlocking surfaces comprises an array of protrusions configured to mate with the array of depressions. The interlocking surfaces preferably form an interference fit when secured with the fastener.

In another embodiment, the proximal section comprises a threaded post, so that a female threaded fastener (e.g. nut) may be used to clamp the link on to the post.

The mounting surface is also configured to provide a temporary attachment for distracting the distance between the first and second vertebral bodies.

In one embodiment, the first platform and second platform are oriented at an angle of inclination with respect to each other to accommodate the natural curvature of the spine segment occupied by the first and second vertebrae.

The link is preferably configured to be selected from a series of links having varying angle of inclination increments.

Another aspect is a method of minimally invasively stabilizing first and second vertebrae in a patient. The method includes the steps of forming a first bone path into the vertebral body of the first vertebrae, forming a second bone path into the vertebral body of the second vertebrae, installing a first bone anchor in the first bone path, and installing a second bone anchor in the second bone path. The first and second bone anchors comprise a proximal mounting surface that allows for positioning of an interconnecting link over the first and second bone anchors to span first and second bone anchors. A first platform of the link is mated with the mounting surface of the first bone anchor, and a second platform is mated with the mounting surface of the second bone anchor. Finally the first and second fasteners are secured to the first and second bone anchors to compress the link on to the first and second bone anchors, thereby restraining motion of the first vertebrae with respect to the second vertebrae.

The first and second bone paths are preferably bored substantially parallel to vertebral endplates of the first and second vertebral bodies. The first and second bone anchors may be delivered to the bored bone path over a guide wire through a central channel running axially along the bone anchor. The link and fasteners may also be delivered over the guide-wire, wherein the cavity is in communication with the central channel.

Another aspect is a method of minimally invasively stabilizing first and second vertebrae in a patient comprising forming a first bone path into the vertebral body of the first vertebrae, forming a second bone path into the vertebral body of the second vertebrae, installing a first bone anchor in the first bone path, and installing a second bone anchor in the second bone path, wherein the first and second bone anchors comprise a distal portion configured to mate with the bone paths and proximal portion comprising a mounting surface. An interconnecting link is positioned over the first and second bone anchors to span first and second bone anchors, mating a first platform of the link with the mounting surface of the first bone anchor, and a second platform of the link with the mounting surface of the second bone anchor; and securing the link to compress the link on to the first and second bone anchors, thereby restraining motion of the first vertebrae with respect to the second vertebrae.

In one embodiment of the current aspect, compressing the link on to the first and second bone anchors comprises deforming at least a portion of interlocking surfaces making up the first and second platforms and the mounting surfaces to form an interference fit between the first and second platforms and mounting surfaces.

In another embodiment, a proximal portion of the bone anchors comprise threaded posts, so that compressing the link on to the first and second bone anchors is done by threading a female-threaded fastener on to each of the treaded posts to clamp the link on to the threaded posts.

Yet another aspect is a bone anchor for establishing a mounting foundation in a vertebra, comprising a distal section configured to be installed into and interface with a bone path bored into the vertebra, and a proximal section comprising the mounting surface, wherein the mounting surface is configured to provide purchase for one or more instruments used in treating the vertebra.

In one embodiment, the proximal section comprises a protrusion coupled to the mounting surface, wherein the protrusion extends from the vertebral body when the bone anchor is installed in the vertebral body.

The mounting surface is preferably configured to allow placement of an interconnecting link over the protrusion, wherein the link is configured to the spanning the bone anchor and a second bone anchor located on a second vertebra to interface with an engagement surface of the link when installed.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a lateral view of the human spine.

FIG. 2 is an upper-posterior view of a human vertebra.

FIG. 3 is a lateral view of two adjacent vertebrae.

FIG. 4 is an anterior view of a patient in preparation for cervical spinal surgery.

FIG. 5 is a view of the cervical spine showing the view of the vertebral bodies seen by the surgeon in an anterior approach.

FIGS. 6A-B illustrate a radiolucent bone anchor guide instrument in accordance with the present invention.

FIG. 7 illustrates a lateral view of the cervical spine with a guide wire installed in the midpoint of a vertebral body from a direct anterior approach.

FIG. 8 illustrates a dilator advanced along the guide wire of FIG. 7 in accordance with the present invention.

FIG. 9 illustrates transparent overlay template having bone anchors of various sizes.

FIG. 10 shows the transparent overlay template of FIG. 9 over radiographic image in accordance with the present invention.

FIGS. 11-12 illustrate exemplary ring bone anchors with a threaded interlock attachment surface in accordance with the present invention.

FIGS. 13-14 illustrate exemplary ring bone anchors with smooth interlock attachment fittings in accordance with the present invention.

FIGS. 15-16 illustrate cross-sectional views of exemplary ring bone anchors.

FIGS. 17-18 illustrate exemplary dowel bone anchors with threaded interlock attachment fittings in accordance with the present invention.

FIG. 19 illustrates a hollow core drill advanced along a guide wire dilator in accordance with the present invention.

FIG. 20 illustrates a tapered drill advanced along a guide wire in accordance with the present invention.

FIG. 21 illustrates a tubular bone path or “kerf” scored into the vertebral body in accordance with the present invention.

FIG. 22 illustrates a tapered bone path scored into the vertebral body in accordance with the present invention.

FIG. 23 shows a ring bone anchor installed in the bone path of FIG. 21.

FIG. 24 shows a dowel implant disposed on an impactor.

FIG. 25 illustrates two bone anchors installed anteriorly on adjacent vertebrae in preparation for interconnection.

FIG. 26 illustrates a lateral view of an vertebral fusion assembly in accordance with the present invention.

FIG. 27 shows the cross-sectional geometry of the interconnect link of FIG. 26.

FIG. 28 shows a mounting surface of the interconnecting link of FIG. 26.

FIG. 29 illustrates a schematic cross-section of a portion of the bone anchor, interconnecting link and locking rivet.

FIG. 30 illustrates an axial view of a bone anchor with a locking rivet installed within the central opening of the bone anchor in accordance with the present invention.

FIG. 31 illustrates an installation tool in accordance with the present invention.

FIGS. 32 and 33 illustrate a locking nut to be used in with the threaded bone anchors of FIGS. 11 and 12.

### DETAILED DESCRIPTION OF THE INVENTION

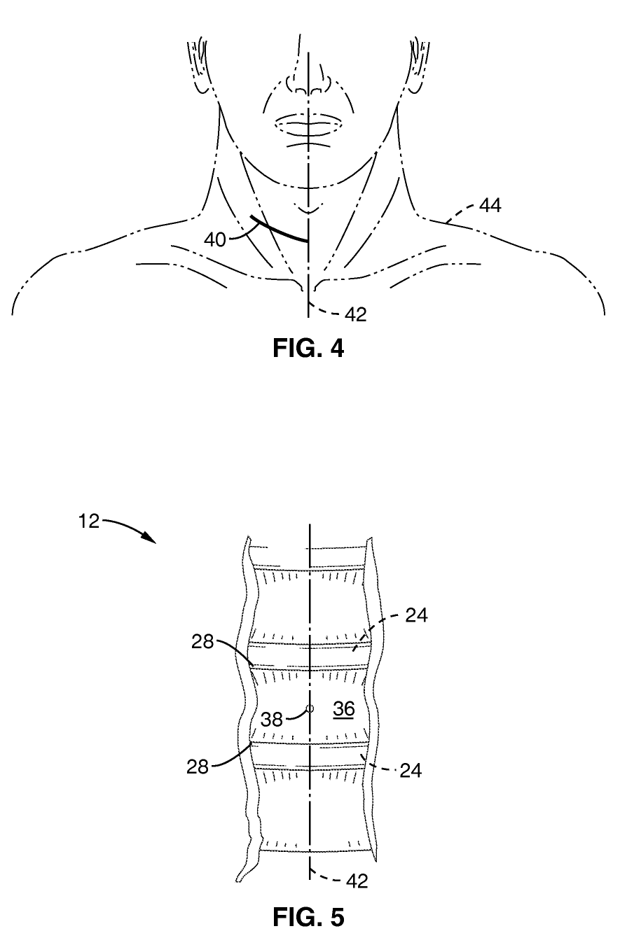

Referring more specifically to the drawings, for illustrative purposes the present invention is embodied in the systems and methods generally shown in FIG. 4 through FIG. 33. It will be appreciated that the systems may vary as to configuration and as to details of the parts, and that the method may vary as to the specific steps and sequence, without departing from the basic concepts as disclosed herein.

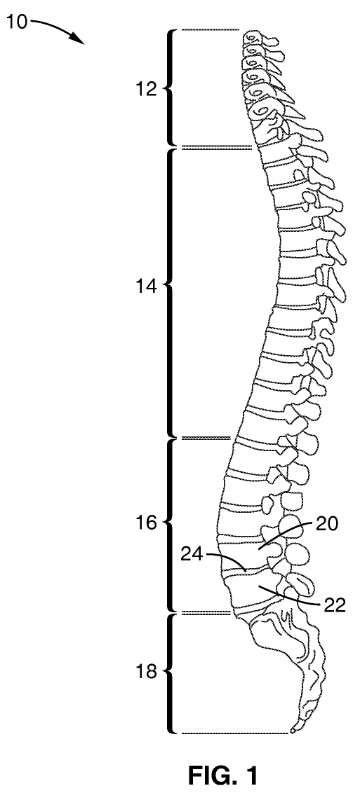

Referring to FIG. 1, a lateral view of the human spine 10 is illustrated showing the various regions of vertebrae: cervical 12, thoracic 14, and cervical 16. The basic biomechanical unit of the spine, referred to as a motion segment, consists of two adjacent vertebrae 20, 22 and the three joint articular complex through which they move and are constrained in relation to one another.

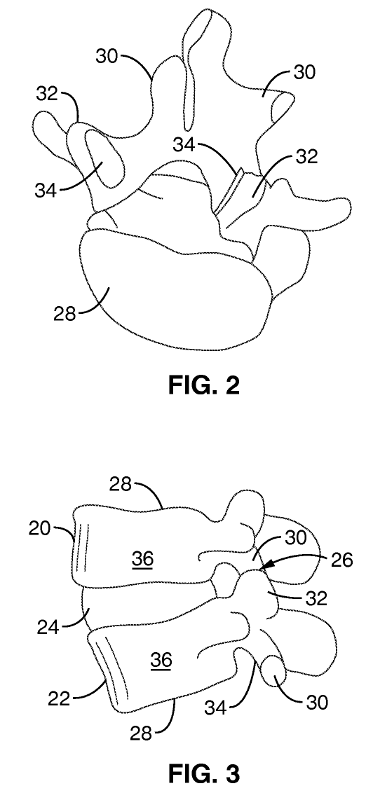

Referring to FIGS. 2 and 3, the cervical spine articulations consist of an intervertebral disc 24 located between the bodies 28 of the adjacent vertebrae 20, 22. Each vertebral body 28 is formed by a shell 46 of hard cortical bone and underlying cancellous subcortical bone 44. The neural elements of the spine are protected by a bony roof called the lamina 30.

The vertebral bodies 28 allow constrained spinal motion while protecting the contained neural structures. In general terms, the intervertebral disc 24 is a viscoelastic universal joint while the bilateral posterior-lateral facet joints 26 are highly constrained sliding planar articulations, lubricated by synovial fluid contained within a joint capsule. The vertebral bodies 36 bear the majority of the axial force transmitted through the spinal motion segment. There are significant differences in the translational and rotational forces imposed on the vertebral bodies in the cervical, thoracic and lumbar spines. In the lumbar spine, the geometry of the lumbar facet joints provides a high degree of resistance to translational shear forces across the horizontal transverse plane of the vertebrae, strong resistance to rotation about the longitudinal (sagittal) axis of the spine, while controlling forward and back bending flexion-extension excursion in the frontal plane and lateral bending rotational motion to within physiologic limits. In the cervical spine, the geometry of the cervical vertebral bodies provides a high degree of protection for the neural elements by limiting normal motion of the spine to within physiologic limits. Ultimately, the stability of the spine 10 is defined in terms of its ability to protect the enclosed neural elements.

FIGS. 4-21 illustrate systems and methods in accordance with the present invention for performing a minimally invasive procedure to stabilize the vertebral bodies 28 between two or more adjacent vertebrae. A procedure on the cervical spine is illustrated for simplicity, although the methods described may be used on the anterior and lateral surfaces of much of the spine, including thoracic, lumbar, and sacral vertebrae. One significant benefit of minimally invasive surgery is the ability to perform the procedure with minimal tissue trauma. Television image intensifier fluoroscopy provides the guidance necessary to allow a surgeon to place instrumentation and bone anchors precisely on the desired anatomic ‘target’. A major point of emphasis of the system of the present invention is the concept of minimizing the impact of the surgery upon the patient's physiology through planned pre-emptive analgesia and minimal tissue trauma.

Referring now to FIG. 4, a cervical surgical procedure is initiated by creating an anterior skin marking 40 on the patient's skin at a short distance lateral to the midline 42 of the patient's neck 44. The marking 40 is preferably made with assistance of fluoroscopic guidance by locating landmarks corresponding to the target region for treatment, such as the vertebral endplates 28 on the underlying target vertebrae 36. An incision is then made along line 40. After the incision is made, standard, microscopically-assisted dissection is carried through the soft tissues of the anterior cervical musculature down to the ventral surface of the vertebral body 36.

Referring now to FIG. 5, a television image intensifier fluoroscopic c-arm (not shown) is precisely aligned with the transverse plane of the superior and inferior end plates 28 of the target vertebrae 36 and the starting point 38 is located on the anterior surface of the vertebral body 36 equidistant between the planes of the two endplates 28. A routinely available Jamshidi bone biopsy needle and trocar (not shown) are then placed at the visible midline 42 of the vertebral body under fluoroscopic guidance.

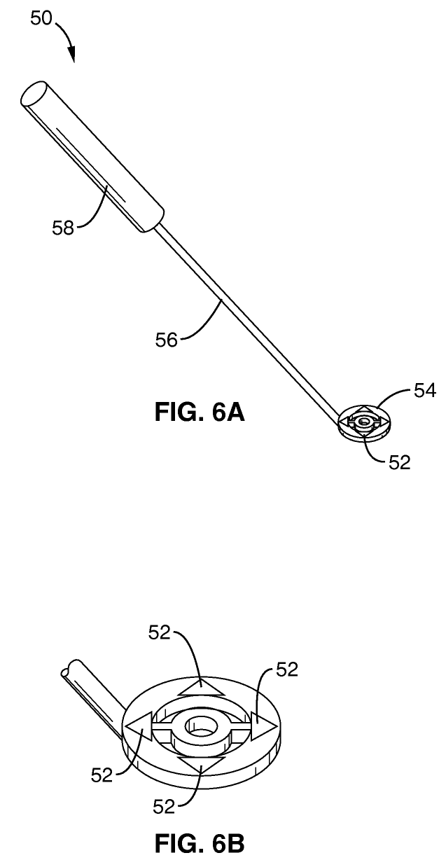

As shown in FIGS. 6A-B, a radiolucent bone anchor guide 50 may then be positioned at vertebral body 36 to determine the size of the bone anchor (described in further detail below) to be implanted. The size of the bone anchor is a function of the patient's vertebral anatomy, e.g. the distance between the superior and inferior endplates 28.

Generally, the larger the bone anchor, the better the stress distribution on the cortical shell and subcortical cancellous bone structure of the vertebral body. Therefore, the patient specific bone anchor size is selected based on the largest configuration that can be accommodated within the bone mass of the vertebral body 28.

As illustrated in FIGS. 6A-B, bone anchor guide 50 has a radiolucent marker 52 on a face plate 54 at the end of arm 56. When placed at the target vertebral body 26, the marker 52 shows up clearly on image intensifier monitor screen to provide a reference as to the available workspace for the bone anchor.

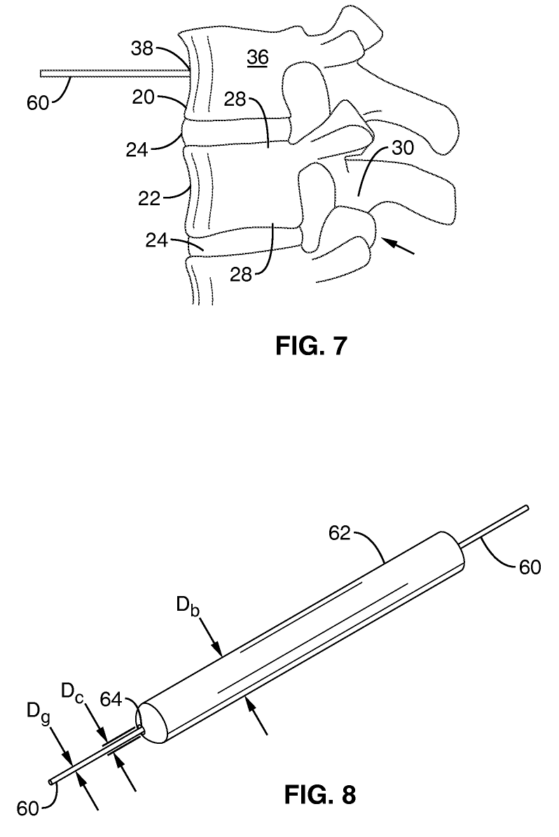

Referring now to FIG. 7, the biopsy needle (not shown) may then be placed in the vertebral body under fluoroscopic guidance. An obturator (not shown) in the center of the needle is removed and a thin stainless steel guide wire 60, e.g. K-wire, is placed in the vertebral body 36 at the target location 38. The guide wire 60 serves as a guide for surgical instruments and the introduction of the bone anchor during the procedure. For example, the guide wire 60 may by used in conjunction with a surgical kit including a range of size-specific drills, inserters, impactors, all of which are cannulated to match to the internal diameter of the instrumentation system correlating to the outer diameter of the custom-length long K-wires 60.

Now referring to FIG. 8, a blunt-end soft-tissue dilator 62 may then be positioned at the target location 38 of the anterior surface of the vertebral body 36 by advancing it with fluoroscopic guidance over the guide wire 60 through a central cannula opening 64 in the dilator. The inside diameter D_{c} of opening 64 is matched to guide wire diameter D_{g} so that the blunt dilator 62 slides freely on the guide wire. The blunt dilator 62 is preferably one of a kit of dilators comprising surgical stainless steel bodies having an outside diameter D_{b} in various increments, e.g. 4 mm, 6 mm, 8 mm and 10 mm, etc.

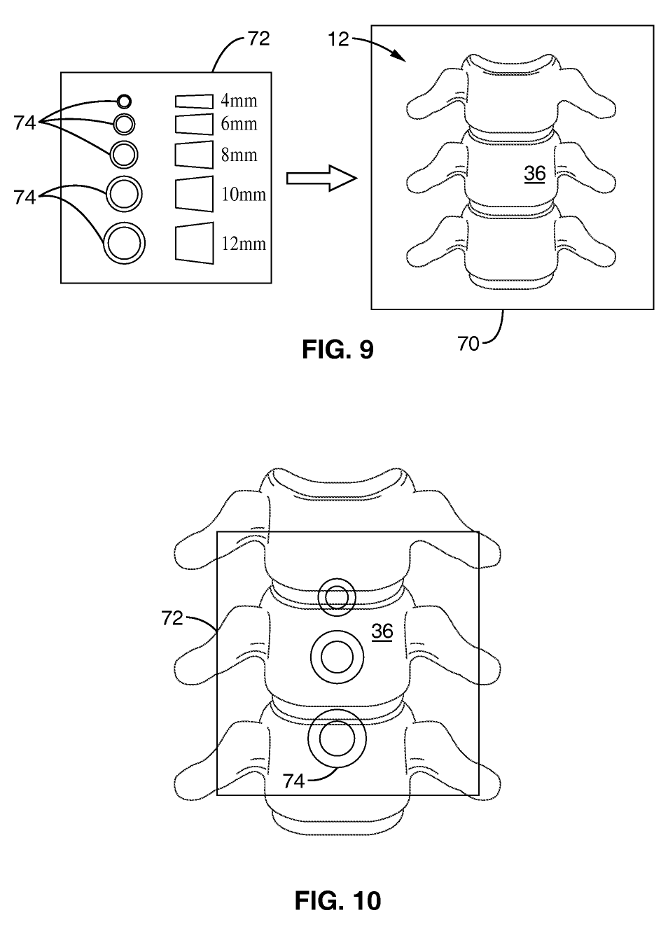

Referring to FIGS. 9A-B, the outside diameter D_{b} of the blunt dilator 62 may be sized initially on the basis of preoperative planning by the surgeon, using a clear plastic overlay template 72 applied to the patient's x-ray, MRI or CT scan images 70. The template 72 has markings 74 which guide the surgeon's choice of the size of the dilator and bone anchor, and is based on measurements of the vertebral body 36. The measurements may be confirmed intra-operatively with the radiolucent bone anchor guide instrument 50 shown in FIGS. 5 and 6.

With the blunt dilator 62 and bone anchor properly sized, the bone path for the anchor may now be scored. Generally, the shape and size of the bone path is a function of the bone anchor selected to be implanted.

Although two types of implants are described herein, it is appreciated that a number of different bone anchors may be used to practice the present invention. Although slight differences in preparation and implantation exist between the different implant types, the biomechanical aspects of vertebral fixation are substantially similar.

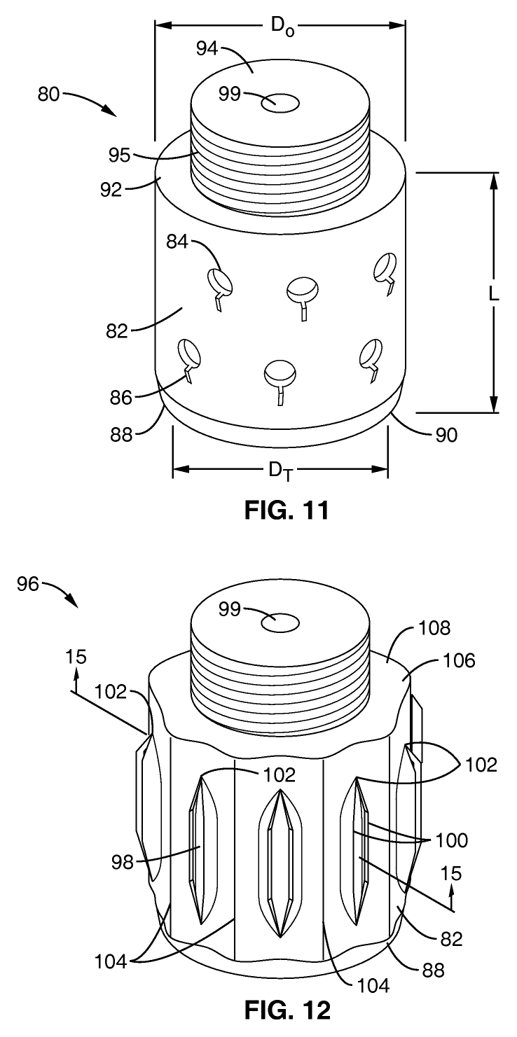

FIGS. 11-16 illustrate several variations embodiments of the ring-shaped bone anchor. Referring now to FIG. 11, ring bone anchor 80 has a distal section 82 comprising a cylindrical tube with walls having thickness T, preferably composed of medical-grade metallic material, such as titanium, or other biocompatible material. The distal section 82 of the ring bone anchor 80 has a plurality of fenestrations 84 to encourage growth of the cancellous bone surrounding and through the installed ring. The leading edge 90 of the bone anchor has a taper 88 of diameter Dₜ to facilitate entry into the circular scored bone defect produced by the drill. The trailing end 92 of the distal section 82 terminates in a proximal section comprising a cylindrical projection 94 (which may be hollowed out from the bottom or leading edge side). The anchor 80 has an axial through hole 99 that allows the implant to be installed over guide wire 60 to the treatment site.

As shown in FIG. 11, protrusion or stem 94 may have a series of threads 95 that form the mounting or interface surface to facilitate a mounting interface between instrumentation (e.g. connection to the soft tissue retraction instrumentation for skeletal distraction of the intervertebral joint), and immobilization of the intervertebral joint when linked with a fixation assembly.

The outer surface of the distal section 82 may be roughened to create or enhance an immediate interference fit with the surrounding bone. For example, a plurality of protrusions 86 may be disposed on outer surface 82 to create an anchoring effect with the surrounding bone. In addition, the outer diameter Dₒ of the bone anchor may be oversized to create an interference fit with the surrounding bone. The bone anchor may comprise various lengths, e.g. ranging from approximately 6 mm to 12 mm, corresponding to the desired depth of the bored bone path. The outside diameter Dₒ may also comprise a number of different diameters, e.g. ranging from approximately 4 mm to 12 mm, also depending on the patient's measured anatomic geometry. The bone anchor 80 may also be one of a kit of bone anchors having increments of e.g. 4 mm, 6 mm, 8 mm, 10 mm or 12 mm outside diameters and lengths of e.g. 6 mm, 8 mm, 10 mm or 12 mm.

Fenestrations 84 may be circular pathways as shown in FIG. 11, or be configured to have one of a variety of shapes. For example, bone anchor 96 illustrated in FIG. 12, may comprise a plurality of columnar fenestrations 98 running axially along distal section 82. Fenestrations 98 may also have raised edges 100 that create an interference fit with the surrounding bone. The ends of the columnar fenestrations preferably converge at tip 102. This axial configuration aides in guiding the bone anchor 96 into the bone path while providing resistance to torque stresses across the vertebral body spanned by the bone anchor, effectively “locking” the vertebral body.

The bone anchor 96 may also comprise a plurality of ridges 104 between each of the fenestrations 98. The ridges are highest at the middle of the band, and taper off toward the ends to form outer diameter 106. These ridges may also be repeated on the inner diameter of the ring to form an interference fit with the other side of the kerf (not shown). The “star-like” longitudinal orientation of the ridges 104 and fenestrations 98 not only facilitate the process of driving the bone anchor into the bone, but also help resist rotational forces from twisting the ring in the bone anchor, as the ridges 104 run perpendicular to the rotational moment created by the vertebral body physiologic stress. The immediate interference fit of the bone anchor is an important design feature which immobilizes the vertebral articulation allowing rapid ingrowth of new blood vessels which support the formation of the new bone which will biologically fuse the bone anchor within the vertebra.

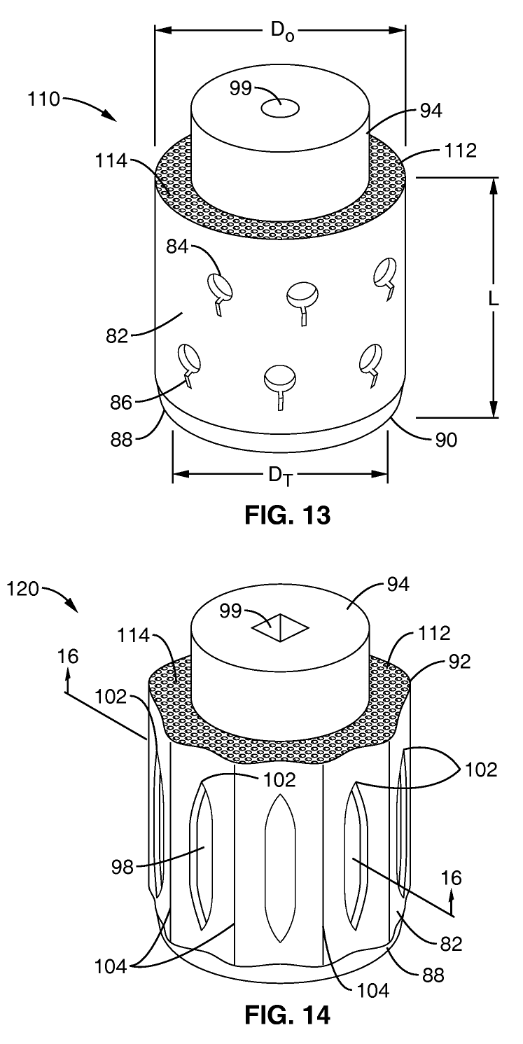

FIG. 13 shows an alternative ring-shaped bone anchor 110. Anchor 110 comprises a generally smooth post 94 and engagement surface 112 having a plurality of indentations 114 that are configured to mate with an opposing surface of a fixation member described in further detail below.

FIG. 14 illustrates another alternative ring anchor 120 having the smooth post 94 and engagement surface 112. The outer surface 82 of anchor 120 is similar to that of anchor 96 shown in FIG. 12, but without the raised edges 100 at the openings of fenestrations 98. This configuration still provides ample rotational resistance and interference with the surrounding bone, while simplifying the configuration for ease of manufacture.

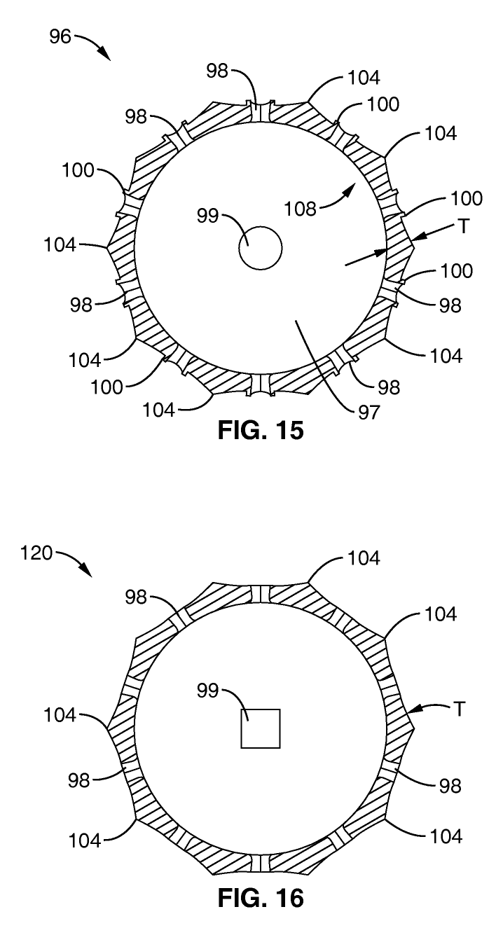

FIGS. 15 and 16 illustrate sections of corresponding implants 96 and 120 to provide an axial view of the bone anchors illustrate the ring thickness T and the papillary interface surface. The central fixation hole 99 locks the bone anchor fixation rivet-implant interface to improve additional resistance to rotational displacement.

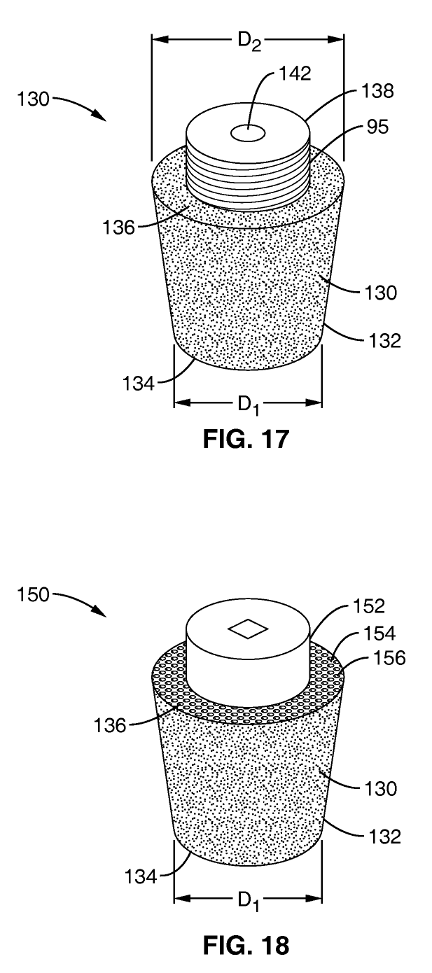

FIG. 17 illustrates a preferred embodiment of a dowel implant 130 in accordance with the present invention. Dowel implant 130 is relatively solid and intended to maximize an immediate interference fit between the roughened outer surface 132 of the implant and the bone of the vertebral body. The implant body is preferably constructed of a porous biocompatible material conducive to bony ingrowth such as nitinol, sintered titanium, tantalum beads or similar material. The dowel implant has relatively central solid core to prevent implant deformation due to crush loading of the implant by the tamp during insertion.

In a preferred embodiment, the implant 130 may comprise or have disposed on the outer surface 132 tantalum beads 134 enhance more rapid bone ingrowth, in to or as an alternative to the tantalum beads 204, the implant may be plasma sprayed with calcium hydroxyapatite to further enhance bone ingrowth.

Preferably, dowel implant is tapered so that leading edge 134 diameter D₁ is smaller than the trailing edge 136 diameter D₂. Although a variety of taper dimensions may be used, the taper typically has a very shallow angle (e.g. 2-3 degrees), or a 0.1 cm diameter decrease per cm of length of the dowel to enhance the press-fit interdigitation between the material of the outer surface of the implant and the rough surrounding cancellous bone in order to resist ‘back-out’ failure of the implant along the insertional axis. The dowel implant 130 may be one of a kit of implants ranging in diameter and length increments, e.g. 6 mm, 8 mm, 10 mm and 12 mm outside diameters with lengths of 4 mm, 6 mm, 8 mm, and 10 mm.

The implant 130 is cannulated with an axial hole 142 having a diameter D_{c} configured so that the implant can be advanced into place over guide wire 60.

Similar to the ring implant of FIG. 11, the dowel anchor 130 may comprise at the trailing end 136 a cylindrical projection 138 that functions as an attachment interface to the stabilization linkage. The dowel anchor 130 protrusion or stem 138 may have a series of threads 95 that facilitate a mounting interface between instrumentation (e.g. connection to the soft tissue retraction instrumentation for skeletal distraction of the intervertebral joint), and immobilization of the intervertebral joint when linked with a fixation assembly.

FIG. 18 illustrates an alternative dowel implant 150 having a generally smooth post 152 and engagement surface 154 having a plurality of indentations 156 that are configured to mate with an opposing surface of a fixation member.

There are a number of possible engineering variants of the implants 130,150 shown in FIGS. 17 and 18 to optimize implant stability, implantation ease and manufacturing suitability. For example, the dowel implants may comprise axial ridges or undulations on the outer surface to maximize resistance to torque-based failure modes.

As with the ring implant, a template system such as that used in FIGS. 9 and 10 may be used during the pre-operative planning process to assess the vertebral body bone mass. With the assessed bone dimensions, the surgeon can determine the largest allowable implant for each vertebral body 36.

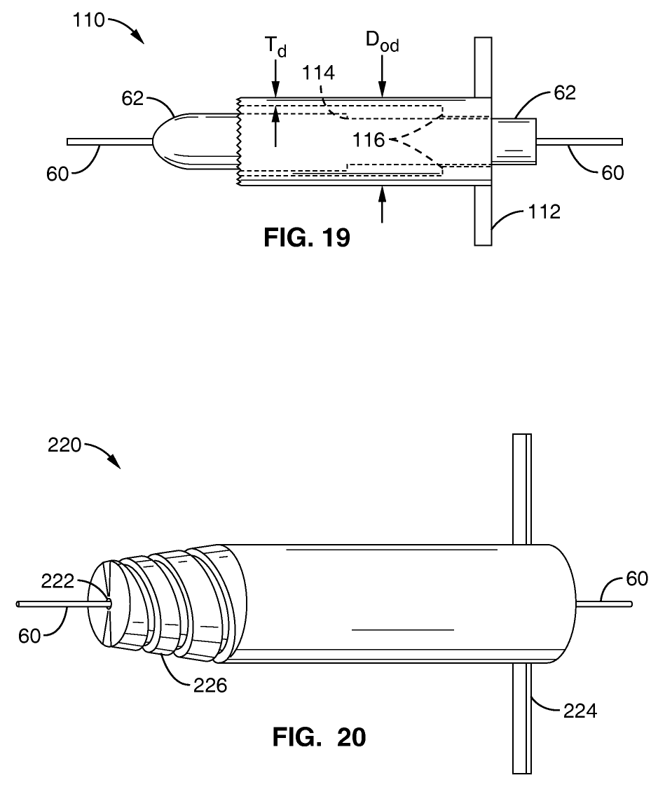

FIG. 19 illustrates a hollow-core drill 110 having a wall thickness T_{d} that may be used to create a scored bone path or kerf for any of the ring-shaped bone anchors illustrated in FIGS. 11-16. The drill 110 may have an inside diameter D_{d} matching the outside diameter D_{b} of the chosen dilator 62 so that the drill 110 may be positioned over and directed by the dilator 62. The outside diameter D_{od} and wall thickness T_{d} of the drill 110 are preferably sized to closely match the outside diameter Dₒ and thickness T of the ring bone anchor (as shown in FIG. 11). For an interference fit, the outside diameter D_{od} of the drill 110 may be configured to match the taper diameter Dₜ of the bone anchor 80.

A ‘T’-handle 112 protruding 90° from the sides of the hollow core drill tube may also be included to allow the surgeon to hand drill the bone around the vertebral body 26. Drill 110 may also be configured with an internal stop 116, such that a notch 114 on the dilator 62 acts to limit the depth of the drill to prevent penetration of the ventral aspect of the combined vertebral body and possible injury to the underlying spinal cord.

FIG. 20 illustrates a dowel drill 220 used to create a scored bone path for a dowel shaped bone anchor such as that illustrated in FIGS. 17-18 advanced over guidewire via lumen 222 to the implant site at the vertebral body 36. Drill 220 has a tapered thread 226 to cut the bone to accept the desired tapered dowel implant 200 diameter. The drill 200 may also have a ‘T’-handle 224 protruding 900 from the sides of the drill tube to allow the surgeon to hand drill the bone.

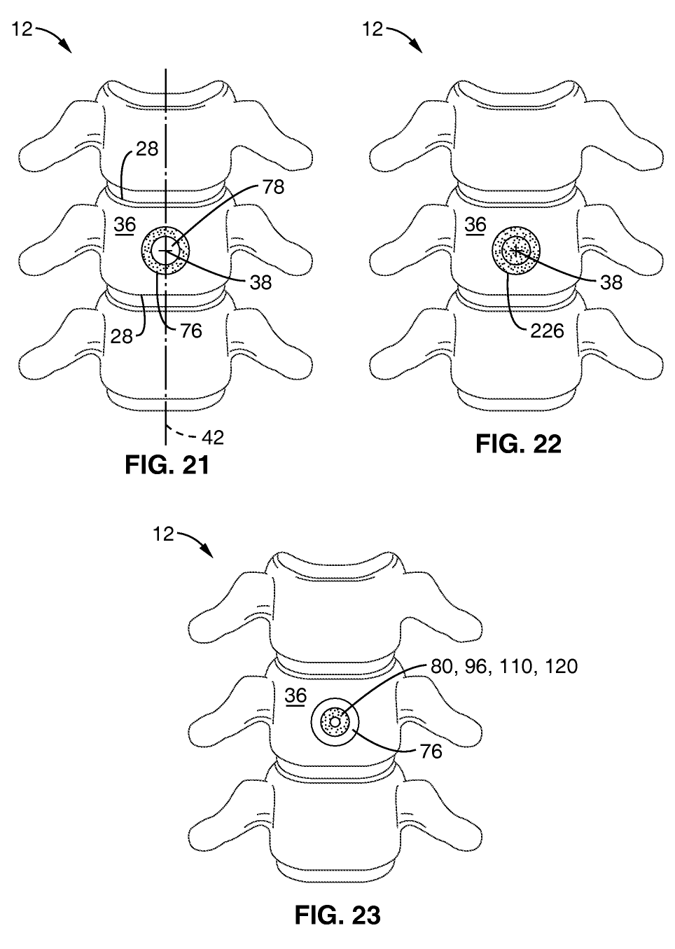

FIG. 21 illustrates a tubular bone path 76 created by drill 110 the vertebral body cortical 46 and subcortical bone 48. The tubular bone path 76 comprises a ring-shaped kerf in the respective vertebral body 36. The bone path 76 is preferably drilled to a depth of approximately 75% of the depth of the vertebral body bone mass. The drilling of kerf 76 leaves a circular bone protrusion 78 in the centrum of the vertebral body 36, i.e. having a central axis coincident with the target location 38.

FIG. 22 illustrates a tapered bone path 226 created by drill 200 into the vertebral body 36 at target location 38. The tapered bone path 226 is shaped to receive dowel-shaped bone anchors 130 or 150 into vertebral body 36. The dowel bone anchors 130,150 are designed to compress and interdigitate with the surrounding bone of bore 226 as it is tapped into place. The circumferential interdigitation of the implant outer surface of the dowel with the spicules of the surrounding cancellous bone provide the necessary stability to protect the bone-implant interface from failure due to applied torque or bending moments.

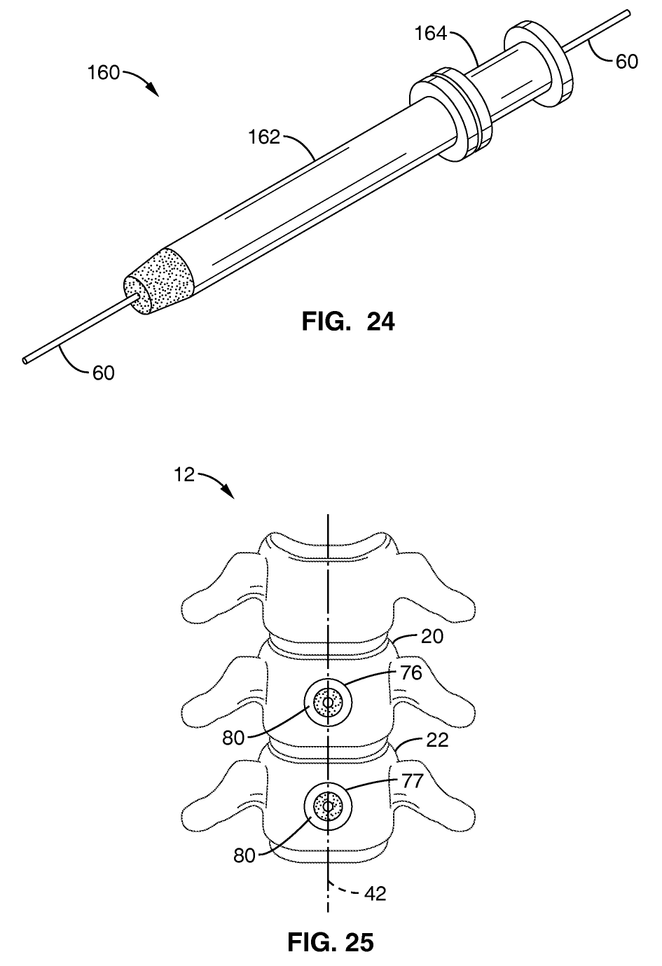

FIG. 23 illustrates a ring-shaped bone anchor, e.g. any of 80, 96, 110 or 120, driven into the bone path 76. An impactor 160, as illustrated in FIG. 24, may be used in conjunction with slap hammer 164, dilator-guide 162 and guide wire 60 to press the ring or dowel shaped bone anchors into the scored bone. With the ring configuration, the fenestrations in the bone anchor allow bone growth from the vertebral body through the walls of the bone anchor to the protrusions 126, 128. This has the effect of locking the bone anchor in place, and results in a stronger interface between the bone anchor and the surrounding bone.

FIG. 25 illustrates an anterior view of the cervical spine 12 with two bone anchors installed in respective kerfs 76 and 77 of adjacent vertebrae 20 and 22. The installed bone anchors now allow the surgeon to use the anchors as the foundation of a soft tissue retraction system, to provide skeletal distraction of the intervertebral joint to facilitate surgery within the disc space and finally to definitively interlock the intervertebral joint.

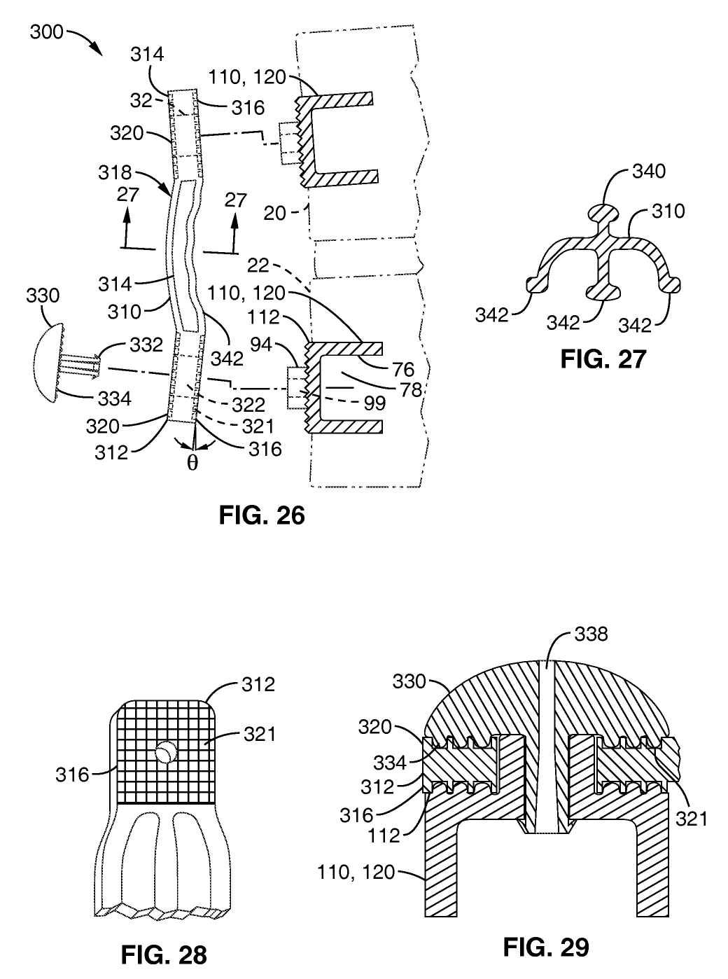

FIG. 26 shows an elevation view of a fixation assembly 300 configured to immobilize motion between adjacent vertebrae 20, 22. Fixation assembly 300 comprises a fixation or interconnecting link 310 having lower platform 312 and upper platform 314 for interface with bone anchors 110, 120. As shown in FIG. 26, the upper platform is offset from the lower platform at an angle θ correlating to the angle of inclination of the vertebral members 20, 22. Angle θ may vary depending on patient anatomy, and vertebral segments being treated.

The fixation assembly 300 is configured to constrain the physiologic motion of the vertebral bodies 20, 22 through the large cross-sectional area of the internal vertebral fixation link 310, which either applies dynamic saggital plane compressive forces to the vertebral bodies or acts as a static bridging device. When assembled, the link 310 neutralizes the predominantly compressive reactive forces of flexion and extension, transferring these strains through the portions of the bone anchors 110, 120 which cross the dense cortical bone surfaces 20 and interlock with the subchondral bone 48 deep within the vertebral bodies.

With the ring and dowel bone anchors of the present invention, reactive forces produced by the day-to-day physiologic loading of the cervical spine are transmitted more uniformly to the column of bone surrounding and encircled by each single bone anchor with a large effective cross-sectional surface area. Wolf's law is a statement of the bone-forming or osteogenic reaction of the cellular elements of bone when subjected to long-term compressive stress. The ring bone anchors of the present invention maximize the translation of the commonly occurring and potentially failure-producing focal, supraphysiologic stresses imposed upon the bone-implant interface into broad areas of compressive strain by stimulating peri-implant bone formation and enhanced biologic fixation.

The vertebral bone anchors 110, 120 are configured to provide a tight immediate interference fit and rapid osteointegration of the bone anchor within the bone. Axial pull-out forces along the insertion axis are effectively resisted by the initial interference fit produced by implant impaction during insertion. During the retraction and distraction functions of the anchors the forces normally ‘seen’ along this axis by the anchors are low.

The bone anchors are also configured to protrude minimally from the vertebral body 20, 22 when installed. As shown in FIG. 26, the bone anchors 110, 120 are sized to protrude slightly (e.g. approximately 1-3 mm) above the natural cupped extent of the vertebral body 20, 22. For even a lower profile, central bone section 78 created from kerf 76 may further be bored down (e.g. to a sub-surface level) to set the anchor deeper in the vertebral body. This may also be done to create clearance for the prongs 332 of fastener 330 described in further detail below.

At the time of final fixation of the construct, the interconnecting link 310 applies a compressive, extension moment to the anchors, dramatically increasing the pull-out strength of the fixation assembly 300. The surface and material properties of the link 310 provide stress relief limiting the risk of fatigue failure of the construct. The link may be manufactured as a set with varying increments (e.g. approximately 1.5 mm length increments and approximately 2°-3° angle of inclination θ increments) to fit precisely between the attachment posts 94 of the vertebral bone anchors 110, 120. The span 318 of link 310 may be bowed to clear the lips of the vertebrae 20, 22 at the vertebral endplates.

FIG. 27 illustrates a cross-sectional view of the span 318 of link 310. The cross section of span 318 may comprise a variety of different shapes and configurations, but is preferably configured to have a low profile while maintaining high bending and torsional rigidity. The span 318 may comprise a plurality of lower arms 342 and upper arm 340 that minimize potential bending of the link 310 when subjected to loading generated by the vertebrae 20, 22. Optionally, the upper arm 340 may be removed to create an even lower-profile configuration.

To lock in the link 310 to the bone anchors 110, 120 a fastener 330 is applied at each bone anchor. For the smooth post-type interface 94 in implants 110, 120 of FIGS. 13, 14, the fastener 330 may comprise a conical connecting cap that is configured to slide over guide wire 60 and into thru-hole 322 in the link 310 and central lumen 99 of the bone anchor. The locking cap 310 may comprise one or more bendable a hook-shaped locking elements 332 that lock to the inside surface of the cylindrical cavity 97 of the tubular bone anchors 110, 120.

The crosslink attachment surface 112 of the vertebral bone anchors 110, 120, the upper and lower attachment surfaces 320, 316 of the link 310 and undersurface 334 of the locking caps 330 comprise a mating ‘pit and dimple’ configuration, shown in further detail in FIGS. 28 and 29. Both the upper and lower mounting surfaces of the 316 and 320 have an array of recesses (e.g. rectangular or hexagonal thin-walled cells that form a waffle-like or honeycomb pattern) that are approximately 1.5 mm deep. The mounting surface 334 of the fastener 330 and upper surface 112 of the bone anchor 110, 120 have a mating series of short (e.g. 1 mm) conical ‘fingers’ that interdigitate with and deform the walls of cells on the opposing surfaces to provide a virtual cold-weld when the locking caps 330 are torqued in place. The random interference fit of these patterns of seemed produce a mating surface with the ability to be fixed at infinitely variable angles of rotation and variable thickness.

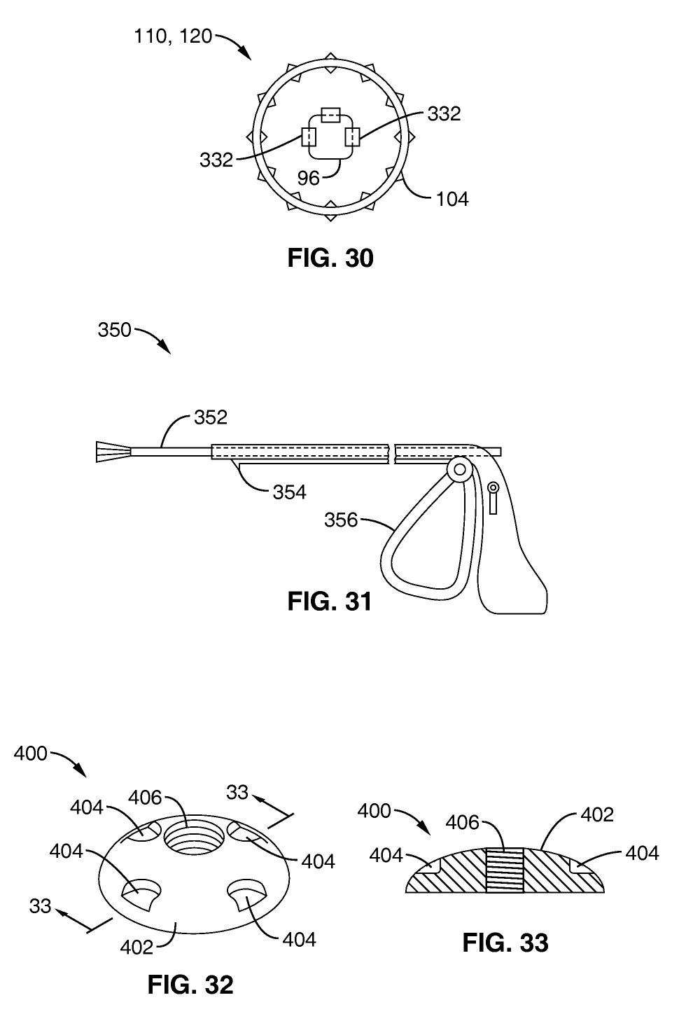

FIG. 30 illustrates a view looking up the bore of bone anchors 110, 120 when the self-locking compressive cap 330 is installed to fasten link 310. The three prongs 332 of fastener 330 are disposed through the link 310 to engage the under surface of the top of the bone anchor 110, 120.

FIG. 31 illustrates an installation tool 350 configured to aid installation of the fastener 330 into the bone anchor. FIG. 22 illustrates an embodiment of the insertion instrumentation. The tool 350 may comprise a central mandrel 352, which may in some applications be used to reinforce the bone anchor, over which the center bore of the fastener 330 is positioned for installation. The tool 350 has a fourth ‘finger’ 354 which is configured to engage with the undersurface of the bone anchor 110, 120 (in the space left open from the three prongs 332 of the fastener 330, and then be pulled back with trigger 356 to compress the frangible titanium honeycomb mating surfaces against one another. Retrieval instrumentation (e.g. a ‘push out’ instrument much like a wheel puller (not shown)) may be used to remove the instrumentation and vertebral body anchors if necessary. Retrieval of the vertebral bone anchors is accomplished with.

FIGS. 32 and 33 illustrate a locking nut 400 to be used in with threaded bone anchors 80, 96 (shown in FIGS. 11 and 12). The nut 400 has a female-threaded central bore 406 that is configured to be threaded over the male-threaded post 94, 108 of bone anchors 80, 96 to fasten down interconnecting link 310. The nut preferably has a smooth, rounded upper surface 402 that forms a low profile when fastened over link 310. The nut 400 may comprise a series of depressions that allow an instrument (e.g. wrench or the like) to torque the nut 400 on to the link 310.

It is appreciated that the system of the present invention may be used as a part of the spine surgeon's reconstructive armamentarium, and may be installed in conjunction with other posterior spine fixation devices such as segmental screws (not shown), to provide more comprehensive stabilization of the cervical spine if required. The bone anchor system of the present invention has broad utility in a number of other clinical situations where it is beneficial to neutralize the forces transmitted across the cervical intervertebral articulations. Examples of systems using anterior cervical plate fixation are disclosed in U.S. Pat. Nos. (6,648,915; 6,010,503; 5,946,760; 5,863,293; 4,653,481), etc., the disclosures of which are incorporated herein by reference in their entirety.

The vertebral bone anchors 80, 96, 110, 120, 130, 150 may also be used as a fixation point for other instruments used in a cervical distraction procedure. For example, retractor blades may be temporarily fixed to the vertebral body anchor mounting interface during the procedure prior to installation of the link 310. The retractor blades may be constructed of radiolucent material to allow fluoroscopic visualization during the case. Plastic inserts (not shown) may be applied to the retractor blades to transmit illumination into the depth of the wound to enhance visualization. The retractor blades may additionally be configured to allow a longitudinal traction force to be applied to the vertebral anchors to provide distraction of the intervening intervertebral joint for interbody graft or bone anchor placement.

It is anticipated that as a result of the minimally traumatic, tissue sparing nature of the anterior cervical fixation system of the present invention, many patients will be able to undergo much less invasive anterior spinal fusion surgery, dramatically reducing the cost and potential morbidity of a these procedures. Secure fixation of the spine allows the patients to avoid the need for a rigid polypropylene cervical orthosis after surgery, lessening muscle atrophy and enhancing rehabilitation. The progress of the patient's fusion is followed with radiographs in the surgeon's office. The preferable materials for fabrication of the bone anchors, such as tantalum, titanium, nitinol, or a bioabsorbable polymer, are compatible with further diagnostic studies using the high magnetic field of an MRI scanner.

Although the embodiments described above are directed to cervical spine fixation, it is appreciated that the bone anchors and interconnecting assembly of the present invention may be used in a variety of locations and procedures in the body. For example, the anchors and link of the present invention may be used to span across a fracture in the patients bone, to stabilize and affect fixation of the fracture instead of commonly used bone screws, plates, and the like.

The system of the present invention provides a robust and rigid mounting interface to provide immobilization/fixation of a treatment site with low-profile and easily installed elements. For example, the bone anchors and interconnecting link may be minimally invasively installed through a small perforation or incision in the patients skin, in contrast to the large incision needed to install bone plates or the like.

Although the description above contains many details, these should not be construed as limiting the scope of the invention but as merely providing illustrations of some of the presently preferred embodiments of this invention. For example, the underlying biomechanical principles of the system and methods of the present invention have a potentially much broader applicability, and may be applied at larger scales to the clinical problems of fusion of other joints and fixation of fractures. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A system for stabilizing first and second body members in a patient; comprising:
a first bone anchor configured to be installed in a bone path bored into the first body member;
a second bone anchor configured to be installed in a bone path bored into the second body member;
wherein the first and second bone anchors comprise a mounting surface;
an interconnecting link configured to be installed on to the first and second bone anchors subsequent to installation of the first and second bone anchors;
wherein the interconnecting link spans the first bone anchor and the second bone anchor when installed;
the link comprising a first platform configured to mate with the mounting surface of the first bone anchor, and a second platform configured to mate with the mounting surface of the second bone anchor; and
first and second fasteners configured to clamp the link on to the first and second bone anchors to restrain motion of the first body member with respect to the second body member.

2. A system as recited in claim 1:
wherein the first body member comprises a first vertebral body;
wherein the second body member comprises second first vertebral body; and
wherein the link is configured to span the first and second vertebral bodies to fuse the first and second vertebral bodies.

3. A system as recited in claim 2, wherein the first and second bone anchors comprise:
a distal section configured to interface with the bored bone path; and
a proximal section comprising the mounting surface.

4. A system as recited in claim 3, wherein the proximal section is configured to protrude from the vertebral body when installed.

5. A system as recited in claim 3, wherein the distal section of the bone anchor comprises fenestrations to promote bony ingrowth from bone surrounding the bone path.

6. A system as recited in claim 3, wherein the distal section of the bone anchors comprise longitudinal protrusions configured to create an interference fit with the surrounding bone path.

7. A system as recited in claim 3, wherein the distal section is tapered to form an interference fit with the bone path.

8. A system as recited in claim 3:
wherein the bone anchor comprises a central channel running axially along the bone anchor; and
wherein the central channel is configured to allow the bone anchor to be installed into the bone path over a guide wire.

9. A system as recited in claim 8:
wherein the distal section is hollowed out to form a tube having a cavity; and
wherein the first and second fasteners comprise a cap configured to be inserted through the central channel and lock to a surface of the cavity.

10. A system as recited in claim 3:
wherein the platform and the mounting surface comprise mating interlocking surfaces;
wherein a first surface of the interlocking surfaces comprises an array of depressions; and
wherein a second surface of the interlocking surfaces comprises an array of protrusions configured to mate with the array of depressions.

11. A system as recited in claim 10, wherein the interlocking surfaces form an interference fit when secured with the fastener.

12. A system as recited in claim 3:
wherein the proximal section comprises a threaded post; and
wherein the fastener comprises a female threaded fastener configured to clamp the link on to the post.

13. A system as recited in claim 3, wherein the mounting surface is configured to provide a temporary attachment for distracting the distance between the first and second vertebral bodies.

14. A system as recited in claim 4:
wherein the first platform and second platform are oriented at an angle of inclination with respect to each other to accommodate the natural curvature of the spine segment occupied by the first and second vertebrae; and
wherein the link is configured to be selected from a series of links having varying angle of inclination increments.

15. A method of minimally invasively stabilizing first and second vertebrae in a patient; comprising:
forming a first bone path into the vertebral body of the first vertebrae;
forming a second bone path into the vertebral body of the second vertebrae;
installing a first bone anchor in the first bone path;
installing a second bone anchor in the second bone path;
wherein the first and second bone anchors comprise a proximal mounting surface;
positioning an interconnecting link over the first and second bone anchors to span first and second bone anchors;
mating a first platform of the link with the mounting surface of the first bone anchor, and a second platform with the mounting surface of the second bone anchor; and
securing first and second fasteners to the first and second bone anchors to compress the link on to the first and second bone anchors, thereby restraining motion of the first vertebrae with respect to the second vertebrae.

16. A method as recited claim 15, wherein the first and second bone paths are bored substantially parallel to vertebral endplates of the first and second vertebral bodies.

17. A method as recited claim 16, wherein installing the first bone anchor comprises delivering the first bone anchor to the bored bone path over a guide wire through a central channel running axially along the bone anchor.

18. A method as recited in claim 17, wherein the link and fasteners are delivered over the guide-wire.

19. A method as recited in claim 15, wherein the bone anchors comprise:
a distal section configured to interface with the bored bone path; and
a proximal section comprising the mounting surface; and
wherein the link and fasteners are secured to the proximal section of the bone anchors.

20. A method as recited in claim 19:
wherein securing first and second fasteners comprises inserting a cap through a central channel in each of the bone anchors and locking a portion of the cap to an inside surface of a cavity of the bone anchor; and
wherein the cavity is in communication with the central channel.

21. A method as recited in claim 15, further comprising:
distracting the distance between the first and second vertebral bodies by engaging the mounting surfaces of the first and second bone anchors;
wherein the distraction is performed prior to installation of the link.

22. A method of minimally invasively stabilizing first and second vertebrae in a patient; comprising:
forming a first bone path into the vertebral body of the first vertebrae;
forming a second bone path into the vertebral body of the second vertebrae;
installing a first bone anchor in the first bone path;
installing a second bone anchor in the second bone path;
wherein the first and second bone anchors comprise a distal portion configured to mate with the bone paths and proximal portion comprising a mounting surface;
positioning an interconnecting link over the first and second bone anchors to span first and second bone anchors;
mating a first platform of the link with the mounting surface of the first bone anchor, and a second platform of the link with the mounting surface of the second bone anchor; and
securing the link to compress the link on to the first and second bone anchors, thereby restraining motion of the first vertebrae with respect to the second vertebrae.

23. A method as recited in claim 22, wherein compressing the link on to the first and second bone anchors comprises deforming at least a portion of interlocking surfaces making up the first and second platforms and the mounting surfaces to form an interference fit between the first and second platforms and mounting surfaces.

24. A method as recited in claim 22:
wherein a proximal portion of the bone anchors comprise threaded posts; and
wherein compressing the link on to the first and second bone anchors comprises threading a female-threaded fastener on to each of the treaded posts to clamp the link on to the threaded posts.

25. A method as recited in claim 22:
wherein the distal portion is hollowed out to form a tube having a cavity; and
wherein securing the link comprises installing a cap over the link and into the cavity to lock the cap to a surface of the cavity.

26. A bone anchor for establishing a mounting foundation in a vertebra, comprising:
a distal section configured to be installed into and interface with a bone path bored into the vertebra; and
a proximal section comprising the mounting surface;
wherein the mounting surface is configured to provide purchase for one or more instruments used in treating the vertebra.

27. A bone anchor as recited in claim 26:
wherein the proximal section comprises a protrusion coupled to the mounting surface; and
wherein the protrusion extends from the vertebral body when the bone anchor is installed in the vertebral body.

28. A bone anchor as recited in claim 27:
wherein the mounting surface is configured to allow placement of an interconnecting link over the protrusion;
wherein the link is configured to the spanning the bone anchor and a second bone anchor located on a second vertebra; and
wherein the mounting surface is configured to interface with an engagement surface of the link when installed.

29. A bone anchor as recited in claim 28, wherein the protrusion is configured to support the link.

30. A bone anchor as recited in claim 26, wherein the distal section of the bone anchor comprises fenestrations to promote bony ingrowth from bone surrounding the bone path.

31. A bone anchor as recited in claim 26, wherein the distal sections of the bone anchors comprise longitudinal protrusions configured to create an interference fit with the surrounding bone path.

32. A bone anchor as recited in claim 28:
wherein the protrusion comprises a threaded post; and
wherein the bone anchor is configured to secure to the link by installing a nut over the threaded post.

33. A bone anchor as recited in claim 28:
wherein the bone anchor comprises a central channel running axially along the bone anchor; and
wherein the central channel is configured to allow the bone anchor to be installed into the bone path over a guide wire.

34. A bone anchor as recited in claim 33:
wherein the distal section is hollowed out to form a tube having a cavity; and
wherein a cap configured to be inserted through the central channel and lock to a surface of the cavity to compress the link to the bone anchor.

35. A bone anchor as recited in claim 26, wherein the mounting surface is configured to provide a temporary attachment for distracting the distance between the first and second vertebral bodies.
